(19) United States
(12) Patent Application Publication
(10) Pub. No.: US 2024/0281207 A1
(43) Pub. Date: Aug. 22, 2024

(54) Intelligent Personal Agent Platform and System and Methods for Using Same

(71) Applicant: Ejenta, Inc., San Francisco, CA (US)

(72) Inventors: Maarten Sierhuis, San Francisco, CA (US); Rachna Dhamija, San Francisco, CA (US); Kyle Benjamin MacNamara, San Francisco, CA (US); Catherine Ann Jenkins, San Francisco, CA (US); Reuben Gaele Jan van der Dussen, San Francisco, CA (US); Chin Hua Seah, Santa Clara, CA (US); Sugreev Chawla, Emeryville, CA (US)

(21) Appl. No.: 18/648,421

(22) Filed: Apr. 28, 2024

Related U.S. Application Data
(63) Continuation of application No. 14/977,585, filed on Dec. 21, 2015.
(60) Provisional application No. 62/096,453, filed on Dec. 23, 2014.

(51) Int. Cl. G06F 7/02; G06N 5/025; G06N 5/043; G06N 20/00; G06Q 10/0639; G16H 50/50

(52) U.S. Cl. CPC G06F 7/023; G06N 5/025; G06N 5/043; G06N 20/00; G06Q 10/06398; G16H 50/50

(57) ABSTRACT

The invention is directed to an intelligent personal agent platform that uses sensor data and other inputs to monitor and detect daily activities, to monitor adherence to goals or plans, and to provide in-context personalized advice, coaching, and support to a user. The platform integrates wearable and environmental sensors that gather real-time behavior data with one or more software-based intelligent personal agents that run on cloud-based or local servers to monitor and analyze the data and provide a response, such as a request to take a certain action. Users can interact with their personal agents wherever they are via a variety of interfaces depending on the communication devices and communication networks that are available to them (e.g. mobile devices, Smart TVs, wearable displays, heads-up displays in a car, touch interfaces, and spoken natural language).

### CROSS-REFERENCE TO RELATED APPLICATIONS

This application is a continuation of U.S. application Ser. No. 14/977,585, filed Dec. 21, 2015, which claims the benefit of U.S. Provisional Application No. 62/096,453, filed Dec. 23, 2014. The entirety of each of the foregoing applications is incorporated by reference herein.

### BACKGROUND OF THE INVENTION

### Field of the Invention

The invention relates to an intelligent personal agent platform and system and methods for using the same, wherein various information regarding a user can be collected, processed, and used to assist the user, autonomously or on demand, in a wide variety of ways. More specifically, the invention relates to a system that utilizes one or more software-based intelligent personal agents, within an intelligent personal agent platform, to analyze collected data about a user, determine whether a responsive action should be taken, to take such responsive action if so determined, and to learn about the user to provide more tailored analysis of the collected data.

### Description of Related Art

Methods and systems for collecting information about a person can be used to track various aspects about the person. For example, a patient's blood pressure can be tracked using a blood pressure monitor, or a person's heart rate can be monitored while the person is exercising. However, such simple systems do not necessarily analyze the data being monitored to determine whether a particular action is required based upon that data. Further, such systems do not collectively monitor other aspects or attributes of the user or integrally analyze the data received from the monitoring of other aspects or attributes of the user to then determine whether a particular action is required in response to such analyses or which specific action should be taken. Further still, such systems do not have the capability to concurrently, sequentially, in real time, or as necessary, collect and monitor data from a variety of different sources, such as machines, sensors (including physical sensors, virtual sensors, and sensors located remote from the user), and other people. Nor do such systems have the capability to provide responsive information to such machines, sensors, or other people or request certain actions be taken by such machines, sensors, or other people. Such systems also do not allow a user to select what information or data is monitored or collected or to establish the purpose (e.g., a goal or plan) for monitoring and collecting such information or data to allow the system to then determine what responsive actions are needed in response to such information or data to achieve the desired purpose. Such systems also lack the capability of learning from the collected data to make predictions or to adjust the action taken in response to such data for a given user. Accordingly, there is a need for a system that has the ability to collect and monitor data from various sources, including, for example, machines, sensors, other people, the environment, etc. and to analyze, learn, and predict such data for the purpose of providing a result or taking an action in response, wherein such action may be requested of a machine, sensor, or other person.

### BRIEF SUMMARY OF THE INVENTION

In general, the present invention relates to an intelligent personal agent platform and system and methods for using the same, wherein various information regarding a user can be collected, processed, and used to assist the user, autonomously or on demand, in a wide variety of ways. More specifically, the invention relates to a system that utilizes one or more software-based intelligent personal agents, within an intelligent personal agent platform, to analyze collected data about a user, determine whether a responsive action should be taken, to take such responsive action if so determined, and to learn about the user to provide more tailored analysis of the collected data.

In some embodiments, the present invention is a system that collects sensor data and other inputs, passes such data to an intelligent personal agent platform that has various services or software modules to monitor and detect daily activities, to monitor adherence to plans and goals, and to provide a response, wherein the response may range from providing feedback to the user or interfacing with another person, machine, or another intelligent agent, including directing such other person, machine, or another intelligent agent to take an action (e.g., to provide in-context personalized advice, coaching, support to a user, information to other agents, machines or people); or making a prediction. The system integrates wearable and environmental sensors that gather real-time data with one or more software-based intelligent personal agents that run on cloud-based or local servers to monitor and analyze the data and provide a response, such as a request to take a certain action or making a prediction. Users can interact with their personal agents wherever they are via a variety of interfaces depending on the communication devices and communication networks that are available to them (e.g. mobile devices, Smart TVs, wearable displays, heads-up displays in a car, touch interfaces, and spoken natural language).

In another embodiment, the present invention provides a system for collecting and monitoring data about a user and for taking an action based upon data analyzed, wherein such system includes at least one user and optionally another person, intelligent agent, or machine; at least one sensor for collecting data about a parameter related to the user; an intelligent personal agent platform for analyzing data from the at least one sensor as well as data received from other persons, intelligent agents, machines, or databases external to the intelligent personal agent platform; and a communications network to facilitate data communications between the user, and optionally another person, intelligent agent, or machine; at least one sensor and the databases; and the intelligent personal agent platform.

In another embodiment, the present invention provides a system for collecting and using information about a user, comprising a first module for collecting a set of data associated with a user; a second module for running a software-based intelligent personal agent comprising a software model having at least one condition associated with at least one rule, comparing the data to the at least one condition to determine whether the at least one condition is met; and providing a response based upon the at least one rule once the at least one condition is met.

In another embodiment, the present invention provides a method for collecting and using information about a user, comprising collecting data from at least one source associated with a user; executing a software-based intelligent personal agent comprising a software model having at least one condition associated with at least one rule, comparing the data to the at least one condition to determine whether the at least one condition is met; and providing a response based upon the at least one rule once the at least one condition is met. In some embodiments, the software model is generated based upon an existing template. In some embodiments, the software model is initially created based upon data collected.

In another embodiment, the present invention provides a method for collecting and using information about a user, comprising collecting data from at least one source to produce collected data; processing the collected data to create processed data that is categorized and associated with the at least one source; providing the processed data to a personal software agent comprising a software model comprising at least one predetermined condition associated with at least one rule; comparing the processed data to at least one predetermined condition to determine whether the condition is met; and taking an action based upon the at least one rule once the at least one predetermined condition is met.

In another embodiment, the present invention provides a computer memory device, comprising instructions stored on said computer memory device for collecting data from at least one source associated with a user; running a software-based intelligent personal agent comprising a software model having at least one condition associated with at least one rule, comparing the data to the at least one condition to determine whether the at least one condition is met; and providing a response based upon the at least one rule once the at least one condition is met.

The present invention has virtually unlimited uses in that the system and methods can be used in many different domains or applications, such as supporting teams of people in the many different domains, such as healthcare, insurance, social networking, military, emergency response, patient monitoring, wellness, automotive, planning, scheduling, navigation, diagnosis, giving advice, support, etc.

### BRIEF DESCRIPTION OF THE SEVERAL VIEWS OF THE DRAWINGS

FIG. 1 is a diagram illustrating the components of a system that includes various sources of data that can be collected or directed to an intelligent personal agent platform according to one embodiment of the system of the present invention;

FIG. 2 is a diagram illustrating the data flow among various components external to the intelligent personal agent platform according to one embodiment of the system of the present invention;

FIG. 3 is a block diagram illustrating the various components and data flows of the intelligent personal agent platform according to one embodiment of the system of the present invention;

FIG. 4 is a block diagram illustrating the hardware and software components of an exemplary sensor for use in the intelligent personal agent platform and the corresponding data flow according to one embodiment of the present invention;

FIG. 5 is a block diagram illustrating the agent service used in the intelligent personal agent platform and data flow between the components of the agent service according to one embodiment of the present invention;

FIG. 6 is a block diagram illustrating the learning service used in the intelligent personal agent platform and data flow between the components of the learning service according to one embodiment of the present invention;

FIG. 7 is a block diagram illustrating the interaction service used in the intelligent personal agent platform and data flow between the components of the interaction service according to one embodiment of the present invention;

FIG. 8 is a block diagram illustrating a user interaction platform for interfacing with the intelligent personal agent platform according to one embodiment of the present invention; and

FIG. 9 is a block diagram illustrating the analytics service used in the intelligent personal agent platform and data flow between the components of the analytics service according to one embodiment of the present invention

### DETAILED DESCRIPTION OF THE INVENTION

The present invention is more fully described below with reference to the accompanying drawings. While the invention will be described in conjunction with particular embodiments, it should be understood that the invention can be applied to a wide variety of applications, and it is intended to cover alternatives, modifications, and equivalents within the spirit and scope of the invention. Accordingly, the following description is exemplary in that several embodiments are described (e.g., by use of the terms “preferably,” “for example,” or “in one embodiment”), but this description should not be viewed as limiting or as setting forth the only embodiments of the invention, as the invention encompasses other embodiments that may not be specifically recited in this description. Further, the use of the terms “invention,” “present invention,” “embodiment,” and similar terms throughout this description are used broadly and are not intended to mean that the invention requires, or is limited to, any particular aspect being described or that such description is the only manner in which the invention may be made or used.

In general, the present invention relates to an intelligent personal agent platform and system and methods for using the same, wherein various information regarding a user can be collected, processed, and used to assist the user, autonomously or on demand, in a wide variety of ways. More specifically, the invention relates to a system that utilizes one or more software-based intelligent personal agents, within an intelligent personal agent platform, to analyze collected data about a user, determine whether a responsive action should be taken, to take such responsive action if so determined, and to learn about the user and provide a more tailored analysis of the collected data.

In some embodiments, the present invention is directed to a system that collects sensor data and other inputs, which in one embodiment is related to a particular user of the system, passes such data to an intelligent personal agent platform having one or more intelligent personal agents and various services or software modules to monitor and detect behavior and daily activities, to monitor adherence to plans and goals, and to determine whether a response is required and to take such response, which may include a wide variety of possible responses, including, for example, taking actions such as providing in-context personalized advice, coaching, and support to a user; causing a person, machine, or intelligent agent to take an action; or making a prediction. Accordingly, it should be appreciated that the system may determine that a response is not necessary in which case, the response is actually no response. Otherwise, the response may range from providing feedback to the user or interfacing with another person, machine, or another intelligent agent, including directing such other person, machine, or another intelligent agent to take an action. For example, the user's interaction with the system allows the user to make use of the data gathered to monitor the user's daily activities, to monitor the user's adherence to any plans or goals that have been established by or for the user, and to receive support in achieving their goals. Following, various embodiments of the system are described in connection with the Figures, including various hardware and software components. In addition, various applications of the system are described.

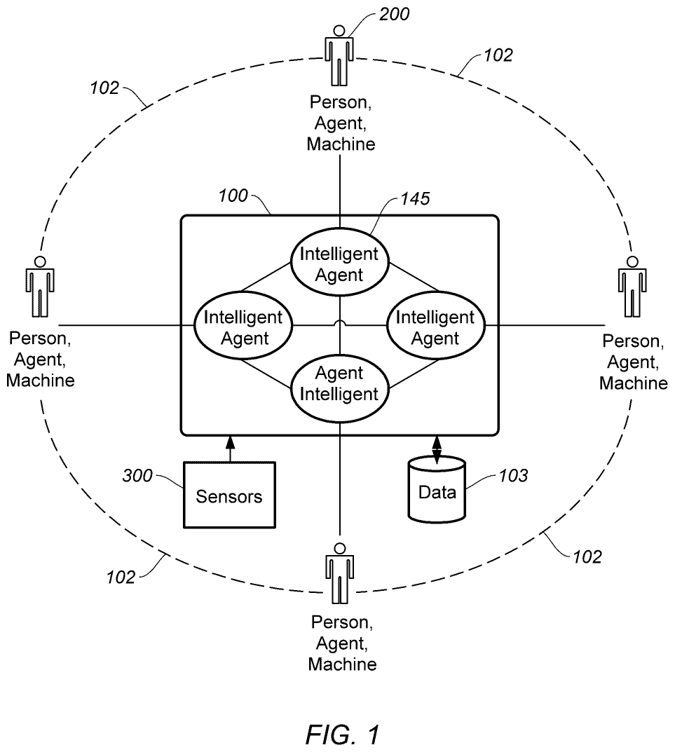

FIG. 1 is a diagram illustrating the components of a system that includes various sources of data that can be collected or directed to an intelligent personal agent platform according to one embodiment of the system of the present invention. Specifically, FIG. 1 is a representation of the overall system, including an intelligent personal agent platform 100 (referred to as the “platform”) having one or more intelligent personal agents 145; persons, machines, and intelligent agents 200; sensors 300; and a generic representation of one or more various databases 103 that are external or, in some embodiments, physically remote, to the platform 100 but from which data can be obtained by the platform 100. FIG. 1 illustrates the interactions between persons, machines, and intelligent agents 200 with each other and with the platform 100 to foster human-human interaction, human-agent interaction, and machine-agent interaction, as well as the interactions between sensors 300 and databases 103 with the platform 100. It should be appreciated that intelligent agents are “social” in that they can communicate, associate, cooperate, and coordinate with other people and agents, and because they take the interests, intentions, or needs of other people and agents into account. It should also be appreciated that one or more of the persons 200 may be a user of the overall system.

In general operation, the sensors 300, the person, agent, or machine 200, and the databases 103 (which may be collectively referred to as external resources), gather data, including real-time data, that is provided to or requested by the platform 100. The platform, specifically the one or more intelligent personal agents 145 within the platform 100, utilize the data obtained from these resources to monitor and analyze the data and provide a response. It should be appreciated that the response can be any response initiated by or taken by the intelligent personal agent 145 and may include a decision to take no action or a decision to directly take an action or make a request for an action to be taken by a separate resource, such as a person, agent, or machine 200 or other internal or external software module or hardware device or machine or person. It should be appreciated that the platform may be operated on cloud-based or local servers with any type of communication with the external resources. Users can interact with their intelligent personal agents wherever they are via a variety of interfaces depending on the communication devices and communication networks that are available to them (e.g. mobile devices, Smart TVs, radios such as land mobile radios, wearable displays, heads-up displays in a car, touch interfaces, and spoken natural language). For example, users can make requests to their respective intelligent personal agents, and after analyzing the necessary data provided by the external resources, the respective intelligent personal agents provide a response. However, it should be appreciated that depending upon the configuration of the intelligent personal agents 145, responses may be provided without a specific request from a user. For example, an intelligent personal agent 145 may be configured to provide a response upon receiving certain data about a user, and the response may be directed to the user specifically or may be directed to an external entity, including a sensor, person, or device or machine. Following, each of the specific components of the system shown in FIG. 1 are described.

The persons, machines, and intelligent personal agents 200 are one or more persons, one or more machines, and one or more intelligent agents that provide data to the platform 100, including data specific to a given user or users. The persons, machines, and intelligent agents 200 are external to the platform 100 but interact with the platform 100 and intelligent personal agents 145 running within the platform 100, for example by providing data input to the platform, including data specific or particular to a given user of the system. However, the persons, intelligent agents, and machines 200 external to the platform 100 are also able to interact with each other outside of the platform 100 as shown by the dashed lines 102. Again, it should be appreciated that one or more persons 200 may be users of the system for whom the intelligent personal agent(s) 145 has been configured, or that person 200 may simply be a person that is part of the overall system and provides data to the system but who is not a user. Accordingly, it should be appreciated that when describing the interactions of a person 200 with the system herein, such may be construed as interactions with a person that is not a user of the system, such as a person that does not necessarily have or use an intelligent personal agent 145, as well as a person who is a user of the system and has or uses one or more intelligent personal agents 145 for the user's benefit.

Sensors 300 provide input or data to the platform 100. The sensors 300 may be any device that basically collects data. For example, the sensors 300 may be physical sensors, virtual sensors, and human services (e.g., feedback from humans) or computational services, such as Siri, Google Now, Amazon Echo, etc. In some embodiments, the system integrates wearable sensors and sensors in the environment (e.g., acoustic, sound, vibration sensors; automotive or transportation sensors; chemical sensors; electric current, electric potential, magnetic, and radio sensors; flow and fluid velocity sensors; ionizing radiation and subatomic particle sensors; navigation instrument sensors; position, angle, displacement, distance, speed, and acceleration sensors; optical, light, imaging, and photon sensors; pressure sensors; force, density, and level sensors; thermal, heat and temperature sensors; and proximity and presence sensors). It should be appreciated that some devices, such as measurement devices, may have more than one sensor. For example, a wireless weight scale that has a pressure sensor that is used to calculate weight, BMI, or body fat, might have a temperature sensor for giving the room temperature or a clock sensor for identifying the time. Further, the sensors may include Internet of Things devices (IoT), like thermostats, door locks, or wellness trackers such as Fitbit or Withings.

The database 103, which may include one or more databases also provide input or data to the platform 100 and include any database that provides or allows access to data that may be used by the system of the present invention. Databases 103 are also external to the platform 100 and may, in some embodiments, be databases that are maintained by third parties or commercial entities that separately from the system of the present invention provide their users with sensors that collect data about a user and store that information in a database maintained by the third party or commercial entity. The system of the present invention then accesses and utilizes the data stored in those third party or commercial entity databases. Examples of such third party or commercial entity databases or services are the Withings service database, Fitbit service database, Foursquare service database, Twitter database, Yelp database, Google Calendar database, and Facebook database, etc.

The platform 100 provides the ability to gather or receive any data, for example, by collecting data from the person, agent, or machine 200, the sensors 300, or the databases 103 or all of these. The data may be collected and processed in real-time (e.g., streaming data) or periodically after it is collected (e.g., batch data). The processing of the data includes its processing to categorize the data and to associate it with a given user of the system or to otherwise prepare the data for use by the intelligent personal agents 145. The processed data is then used by the one or more intelligent personal agents 145 within the platform 100. Each intelligent personal agent 145 applies a given software model having at least one or more rules and one or more conditions concatenated by “ands” and “ors” to determine whether and what response is to be produced. For example, the intelligent personal agent 145 will determine based upon the data it has available to it from the various resources within and outside of the platform 100, whether the condition or conditions for its rule or rules has or have been met. Once such conditions have been met, the intelligent personal agent 145 will apply the rule or rules to determine what response should be taken. It should be appreciated that the conditions may be conditions that are predetermined and set by the user or set for the user, depending upon the model being used. Further the conditions may be conditions that are changed over time by the user or may be changed over time depending upon an analysis of the user's data. It should be appreciated that intelligent personal agents 145 can interact with other intelligent personal agents 145 inside the platform 100, and they can interact with people, other machines, and other intelligent agents 200 outside of the platform 100. A user (e.g., one of the persons 200) interacts with the platform through one or more intelligent personal agents 145 specific to that user. It should be appreciated that a given user may have more than one intelligent personal agent 145.

The platform 100 can be operated on a variety of computing hardware and software components, such as networked computers and data servers (which can be physical machines or virtual machines) and on mobile computing devices, such as smart phones and tablets, smart TVs, and smart watches. For example, in one embodiment, the platform 100 runs on one or more cloud-based virtual servers (e.g., such as those available through the Amazon Web Services cloud platform or the Microsoft Azure platform). The sensors 300 may be a user's smartphone that is running a sensor client application 320 (described further below) that sends sensor data from the sensors (e.g., GPS, accelerometer, gyroscope sensors) on the mobile phone 310 (described further below) directly to the platform (via the sensor service 110 described further below). The user may be wearing a wearable sensor device (e.g., a wrist worn activity tracking device) that sends sensor data via another sensor client 320 running on the user's smartphone to a third party data database 103 outside of the platform 100. In this case, the sensor service 110 may retrieve the activity tracking device sensor data from the third party database 103 instead of directly from the wrist worn activity tracking device. It should be appreciated that each of the components of the system described herein may be referred to generically as a module, and may be embodied as a piece of hardware, such as a physical sensor, or as software, such as a set of software instructions or software application. Further, it should be appreciated that the system components or modules may be connected through a computer network that has one or more computing devices that include processors, processing circuitry, memory, and software instructions for operating the computing devices.

The intelligent personal agent 145 is an artificial, non-human, software component with which humans or other intelligent agents, both inside and outside of the platform 100, can interact as if it is an independent behavioral entity. In some embodiments, the intelligent personal agent 145 is a software model and may be a software model of virtually anything, including, for example, any person, system, machine, action, activity, or process, wherein the software model includes one or more rules and one or more conditions, as described above. It should be appreciated that a given model may be selected from the domain template database 180, which holds a variety of template that may be used as models (described further below), or the model may be created based upon data about a given user by the learning service 130 (described further below). Generally, the intelligent personal agent 145 takes the data or processed data from the external resources and applies the one or more rules to determine if the one or more conditions have been met and to then provide a response as specified by the one or more rules.

The intelligent personal agent 145 can be developed and executed within the platform 100. In the field of artificial intelligence, an intelligent personal agent is a software agent that is an autonomous entity that observes through sensors and acts upon an environment using actuators and directs its activity towards achieving goals (i.e. it is rational and uses knowledge to achieve their goals). Accordingly, the intelligent personal agents 145 can be used to process data and to produce a result or request or that cause an action to be taken, which may include directing, causing, or requesting that another entity, including, for example, a person, system, machine, or other intelligent agent, to take a specific action or not. Accordingly, intelligent personal agents 145 can communicate with various entities. Intelligent personal agents may also learn and may include a model of human behavior to reason and act. In the platform 100, intelligent personal agents 145 can be developed, for example, through the use of the Brahms Agent Language, which is available from Ejenta, Inc.

More particularly, intelligent personal agents 145 can be developed with several attributes and capabilities. Intelligent personal agents 145 have the capability to communicate with people, other intelligent agents, systems, and machines. Users can interact with their respective personal intelligent personal agents via various user interface mechanisms, including web interfaces, mobile devices (phones, tablets, watches), Smart TVs, radios such as land mobile radios, touch screen displays, heads up displays, and spoken natural language, text message, and chat platforms. Intelligent personal agents 145 can communicate with other agents and with devices, machines, services and other systems via application programming interfaces (APIs), or other communication protocols and standards. Communication with an intelligent personal agent 145 can be bilateral (person to agent, agent to person, or agent to agent) or multi-lateral (many people to many agents, many agents to many people, or many agents to many agents).

Further, intelligent personal agents 145 can monitor humans and systems using captured sensor data (e.g., monitor physiological metrics, movement, location, proximity, vital signs, social interactions, calendar, schedule, system telemetry). Intelligent personal agents 145 can analyze data by receiving data from any input such as users, persons, agents, and machines 200; sensors 300; and other agents 145 within the platform 100, and run analytic algorithms to calculate high-level information about the users, systems, and environment that are being monitored. It should be appreciated that analysis can be performed on past sensor data or current data that is being collected in real time. The analyses may include: review or playback of data that occurred in the past; simulation of behavior to predict behavior from current or past data; detection, inference, and learning of higher level activities and patterns of behavior (e.g., detect if someone is currently eating or drinking, infer that the person is eating lunch, and to learn or predict what time they usually have lunch); or personalize or update the agent's model of the user or system, based on observed sensor and behavior data and machine learning algorithms applied to that data (e.g. learning a user's locations and activities and predicting the user's schedule of activities and locations, given his or her current location, activity and time).

In addition, intelligent personal agents 145 may ask the user questions or answer questions from the user based on the knowledge the intelligent personal agents 145 have, which may include knowledge derived from the analytics performed by the intelligent personal agents 145 or from databases, such as external databases 103. In particular, intelligent personal agents 145 can provide advice, feedback, alerts, warning, reminders, or instructions to users and other agents during an activity, based on situational and contextual information, including information about roles and organization of agents and people, activities, sensor data, location, plans, or schedules and calendars. Such are examples of the response that may be made by the intelligent personal agents 145.

Intelligent personal agents 145, as further examples of the response that may be made, may also take actions in the real world (e.g., to order transportation for a user through a transportation service or to turn an appliance on or off) or virtually in software (e.g., to send an email or a text message to another user). Intelligent personal agents 145 may automate tasks or serve as the proxy for a participant in a particular activity. Intelligent personal agents 145 may automatically create a plan and/or schedule of activities based on goals or objectives that are input by the user. Intelligent personal agents 145 can determine how users (or systems) conform, adhere, or deviate from plans or schedules. Intelligent personal agents 145 can coordinate with other people, systems, or agents (e.g., to schedule a meeting at a time that all participants have availability on their schedules and to send all parties the relevant information needed for the meeting). It should be appreciated that all of the tasks capable of being performed by an intelligent personal agent 145 can be done when instructed by the user, another intelligent agent, or by following a plan that is provided to the intelligent personal agent 145.

It should be appreciated that non-intelligent agents, i.e. “actors” that are not goal-directed and do not have a reasoning capability, may also be used within the platform 100. Non-intelligent agents or actors operate on pre-specified conditions. For example, a non-intelligent agent may be used to receive a message from one component of the platform 100, translate that message as necessary, and send it to another component, such as an intelligent agent. A non-intelligent agent can be developed with actor computer languages, such as Java or AKKA.

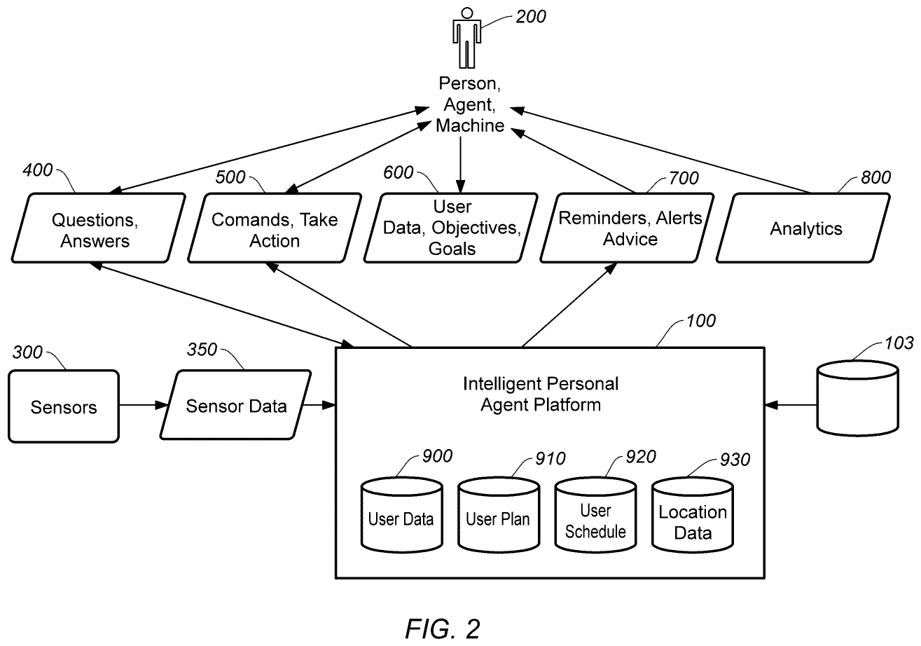

FIG. 2 is a diagram illustrating the data flow among various components external to the intelligent personal agent platform according to one embodiment of the system of the present invention. The intelligent personal agent platform 100 receives and sends data to various components external to the platform 100, including one or more sensors 300, as well as to one or more persons, intelligent agents, and machines 200. The sensors 300 generate sensor data 350 that is sent from the sensor and received by the platform 100 for processing. The person, intelligent agent, or machine 200 generates outputs that are received as inputs by the platform 100 and also receives outputs from the platform 100 as inputs to the person, intelligent agent, or machine 200. Communication to and from the platform 100 are handled through messages referred to as “communicative acts.” These messages include the sender and receiver of the message and the subject and data of the message. The communicative acts can be sent via various formats such as XML or JSON using various protocols such as HTTP or RabbitMQ.

As noted in connection with FIG. 1, sensors 300 may be physical sensors. Physical sensors may be any hardware device that can detect a change in any parameter, such as a change in an event or changes in quantities, and that provides a corresponding output related to the change, such as an electrical or optical signal. Physical sensors may be stationary or mobile and may be located anywhere as required to detect a change in the parameter being measured. Physical sensors may be embedded in devices that are (i) worn by users (e.g., activity trackers, smart watches, smart clothing, smart patches and tattoos, smart contact lens), (ii) embedded in the devices carried by or used by users (e.g., mobile phones, tablets, blood pressure monitors), (iii) contained in the human body (e.g., smart ingestible pills, sensors implanted in the skin or body), (iv) embedded in the environment (e.g., air quality sensors, bio-chemical sensors, sensors in the car, sensors in the home, sensors outside, sensors in roads, traffic lights), and (v) embedded in everyday objects a user may interact with (e.g., furniture, appliances, toys, weight scales, thermostats, door locks, smoke alarms, batteries, household utensils).

Sensors 300 may also be virtual or software-based sensors and may include, for example, software in which several measurements are processed together or where measurements or process parameters from one metric are used to calculate another metric or to estimate the quantity of interest. These sensors may include: (i) multiple sensor data metrics that are used to calculate a new metric (e.g., orientation may be calculated by using measures from a gravity and geomagnetic sensors) and (ii) data from software or third party services (e.g., calendar, social networks like Twitter, Facebook) used to supplement physical sensors or when physical sensors are not available (e.g., to determine who is in proximity using a calendar data, when proximity sensors are not available).

In some embodiments, the person, intelligent agent, or machine 200 can exchange various inputs and outputs 400, 500, 600, 700, 800 with the platform 100. For example, the person, intelligent agent, or machine 200 can make requests 400 to the platform 100 and respond to questions 400 from the platform 100. Similarly, the platform 100 can make can make requests 400 to the person, intelligent agent, or machine 200 and can respond to questions 400 from the person, intelligent agent, or machine 200.

The person, intelligent agent, or machine 200 can issue commands or instructions 500 to the intelligent personal agents 145 running inside the platform 100 to require the intelligent personal agents 145 to perform an action, or the intelligent personal agents 145 inside the platform 100 may automatically take an action themselves (e.g., a user can instruct the intelligent personal agents to turn on a light, or the intelligent personal agents 145 may turn on a light automatically without instruction). Actions may be performed in the real world or in the virtual world (e.g., executed in software). The intelligent personal agents 145 inside the platform may also instruct the person, intelligent agent, or machine 200 to perform an action.

The person, intelligent agent, or machine 200 may provide user specific data, objectives, or goals 600 to the intelligent personal agents 145 running inside the platform, and vice versa. The person, intelligent agent, or machine 200 may also provide reminders, alerts, advice (e.g., a suggestion or recommendation) and coaching (e.g., a set of instructions to perform an activity or to achieve a goal) 700 to the intelligent personal agents 145 running inside the platform 100, and vice versa. Further, the intelligent personal agents 145 running inside the platform 100 can provide analytics to the person, intelligent agent, or machine 200 outside of the platform 100.

It should be appreciated that the intelligent personal agents 145 within the platform 100 may also exchange data inputs and outputs with various databases 900, 910, 920, 930 that are included in the platform 100. User data maintained in a user data database 900 includes data that describes the user, such as account information, personal information, contact methods, and identification information about the sensors, devices, persons, and third party accounts that are associated with the user. Third party accounts include accounts the user may have with other third party services, such as Skype, Google, or Twitter. It should be appreciated, however, that the user data database 900 only stores account information about the user's third party account, the user's data generated through use of that third party service is still stored with that third party. For example, if the user wants to be able to tell his agent to make a Skype call or to be able to add an appointment on his or another person's Google Calendar, information about those accounts is stored in the user data database 900. The user's Skype contact list and phone numbers and Google Calendar data, however, are stored in a database 103 external to the platform 100 that is operated or controlled by the respective third party. Specifically, sufficient information about these third party accounts is stored to allow receipt of the user's credentials for those accounts or to allow use of protocols like OAUTH that give the platform 100 authorization tokens that allow the platform 100 to access or to take actions on behalf of the user in connection with those third party services. Data about a user's plan is maintained in a user plan database 910. The user plan data includes a list of tasks or actions with timing, due dates, or deadlines and resources necessary to achieve a defined objective or goal for the user (e.g., take a particular medication once per day with food, lose weight by a certain date) or a group of users, such as a group project or mission (e.g., a health provider whose goal is to reduce hospital readmissions in its patient population). Data about a user's schedule is maintained in a user schedule database 920. A user's schedule contains a list of times at which possible tasks, events, or actions are intended to take place, or a sequence of events in the chronological order in which such events are intended to take place (e.g., take medication at 12 pm every day with lunch). A schedule can be created or modified by the user or by the intelligent personal agent 145 within the platform 100. Data about the location of various items related to the user are maintained in a location database 930. Location data describe indoor or outdoor logical or conceptual locations (such as latitude and longitudinal coordinates, geography descriptions, or proximity to sensor devices, or Wi-Fi access points or cellular towers), which may be labeled with a name. The intelligent personal agents 145 within the platform 100 may utilize data from any one or more of these databases 900, 910, 920, 930 for purposes of performing whatever task the intelligent personal agent 145 is performing.

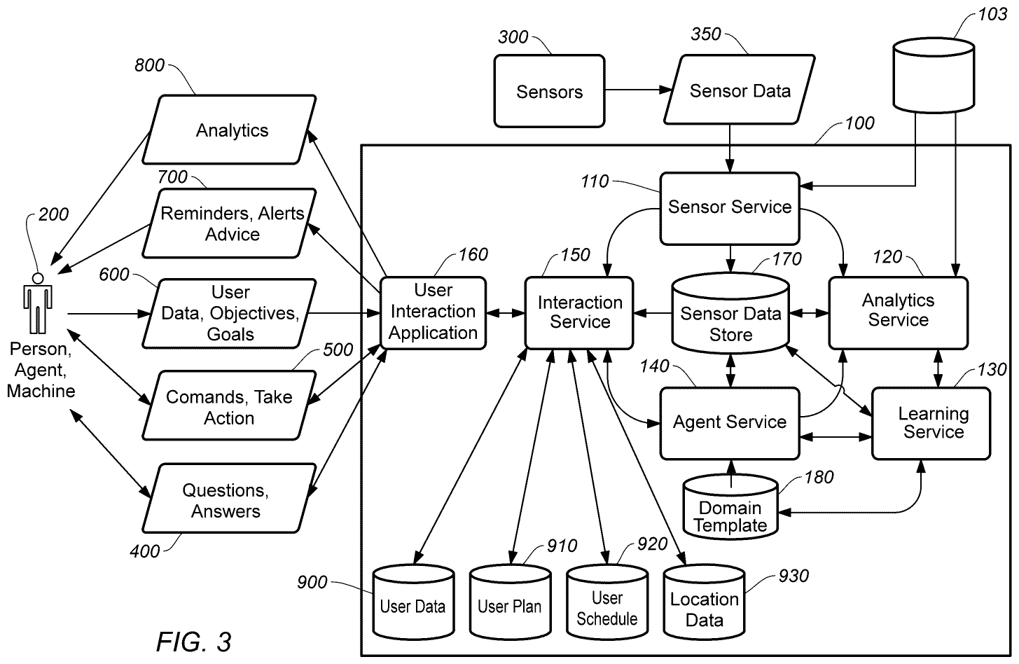

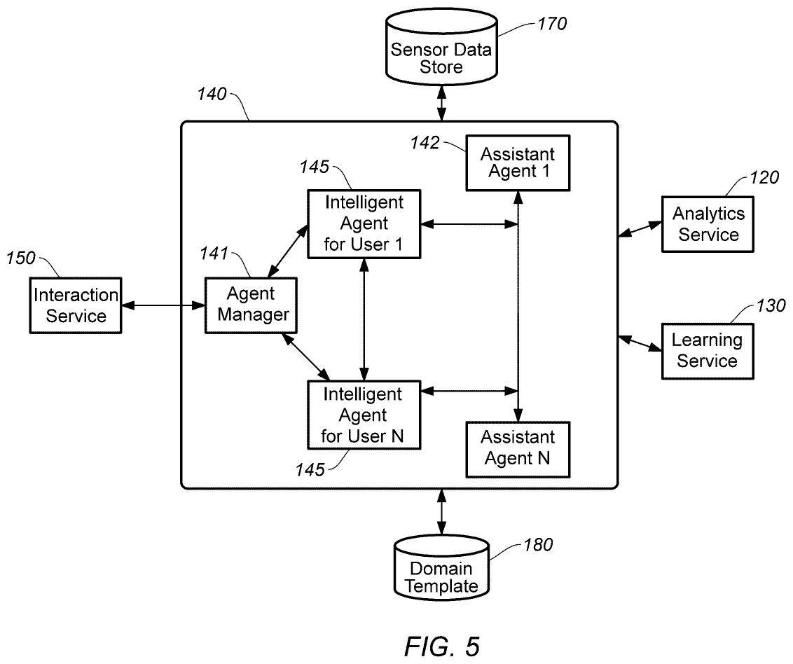

FIG. 3 is a block diagram illustrating the various components and data flows of the intelligent personal agent platform according to one embodiment of the system of the present invention. Within the platform 100, there are several components that perform various functions or services, including the sensor service 110, the analytics service 120, the learning service 130, the agent service 140 (within in which resides the intelligent personal agents 145 and which runs the intelligent personal agents 145), the interaction service 150, and the user interaction application 160. In addition, additional components within the platform 100 include the sensor data store database 170 and the domain template database 180. Again, the agent service 140 is the service that runs the intelligent personal agents 145 (not shown in FIG. 3) within the platform 100 and is described in more detail in connection with FIG. 5 below.

It should be appreciated that each of these services may communicate with one another as necessary. Generally, the sensor service 110 communicates sensor data to the platform 100 and various other services; the interaction service 150 communicates data from the user (e.g., user settings, preferences, user data, communications to/from other users) to the platform 100 and various other services; the agent service communicates inferred knowledge about and actions for the user, person, intelligent agent, or machine, and the activity and context to various services; and the analytics service 120 communicates information derived from analyzing one or more streams of data. Each of the services can communicate directly with each other, or via one of the other services.

The sensor service 110 comprises software that receives sensor data 350 from one or more sensors 300 as well as from external databases 103 and stores this data in the sensor data store database 170, which is a database for efficiently storing sensor data, including streaming sensor data. It should be appreciated that the sensor service 110 may also communicate with the analytics service 120 and the interaction service 150 to provide the sensor data to these services as needed.

The analytics service 120 comprises software that receives sensor data from the sensor data store database 170 as well as from external databases 103 that needs to be analyzed. The analytics service 120 enables different analysis tasks for each type of sensor data or data from databases 103 and allows users (or data analysts) to analyze multiple sensor data streams and data from the databases 103 at the same time. It should be appreciated that any number of analyses may be performed, including, for example, sensor data fusion, historical analysis, descriptive statistics, correlations, feature aggregation, trend analysis, and machine learning. In other words, any analytical analysis algorithm can be programmed or used as needed. Results of the analysis performed by the analytics service 120 are stored in the sensor data store database 170. It should be appreciated that the analytics service 120 may also receive data from the sensor service 110 directly to facilitate the generation of analytics on the sensor data and from the agent service 140 to also facilitate the generation of analytics based on information from the intelligent personal agents.

The learning service 130 is a service that takes input from the agent service 140, the analytics service 120, and the sensor data store database 170 and learns about a user's individual behavior as described by features (measures or parameters that are being observed by one or more intelligent personal agents 145 within the platform 100 via sensors, via the analytics service, or other inputs). The learning service enhances or updates a user's domain template in the domain template database 180 with more individualized or specific knowledge about the user. The user's domain template is a computer model that describes domain-specific rules, activities, actions, communication, attributes, beliefs, or conditions for each of the supported user roles in a given domain or application and forms the basis for operation of the model(s) used by the intelligent personal agents 145. For example, in a domain of patient monitoring, the user may be in the role of the patient versus a caregiver. In this situation, the domain template would be those rules, etc. that apply to a patient in a patient monitoring domain. Intelligent personal agents 145, as described in more detail below, may perform one of more of these user roles and inherit the rules, activities, communications etc. for each such role. The learning service 130 is able to change the domain specific rules from a given user's domain template by changing the beliefs and conditions of the rules to generate more individualized or specific rules based on past examples and thereby provide a more individually specialized model for the intelligent personal agent 145 used by that user in that domain. It should also be appreciated, as described further below, that the learning service 130 may also be used to create a model for a given user or users that is then used by a respective intelligent personal agent(s) 145, rather than using an existing model, such as a model based upon a template in the domain template database 180, noting that such created model may be stored in the domain template database 180 for later use as a template. Accordingly, the learning service 130 communicates with the agent service 140, the analytics service 120, the sensor data store database 170, and the domain template database 180.

It should be appreciated that, as noted, the learning service 130 takes in data from specific examples and is able to generalize the user's behavior from these examples. These generalizations can be, for example, data about past activity behavior that is stored into the sensor data store database 170 for use by the agent service 140 in the future. If this data is immediately relevant to one or more given intelligent personal agents 145, it can also be communicated directly to those intelligent personal agents 145 in the agent service 140 for immediate use. This way the intelligent personal agents 145 do not have to retrieve it from the sensor data store database 170. Accordingly, it should be appreciated that the learning service 130 can both send information to, and receive information from, the sensor data store database 170, as well as the agent service 140.

The interaction service 150 is the service that manages the interaction between the user and the platform 100. The interaction service 150 communicates with the user interaction application 160, which is an application running on a device (e.g., phone, tablet, radio such as land mobile radio, computer, watch, TV, car) that enables a user to interact with it to enter input and receive output, for example, via a GUI (graphical user interface), touch display, keyboard, mouse, gesture, or voice interaction. The interaction service 150 can receive communications and data from the sensor service 110, the sensor data store database 170, as well as any one or more of the various databases 900, 910, 920, 930 that are external to the platform 100, as described above in connection with FIG. 2. The interaction service 150 also communicates with the agent service 140. The interaction service 150 communicates with these various components within and external to the platform to pass data as necessary between these components.

With respect to the interaction between the interaction service 150 and the agent service 140, it should be appreciated that when the agent service 140 executes an intelligent personal agent 145 within the platform 100, the intelligent personal agent 145 may require data from the various external components of the system (via the user interaction application 160), such as the persons, intelligent agents, or machines 200 or databases 103 that are external to the platform 100. Similarly, the intelligent personal agent 145 may require data from the various internal components within the platform 100, such as the various databases 900, 910, 920, 930, the sensor service 110, or sensor data store database 170 to perform its task. The agent service 140 may receive data directly from the user or may receive data about the user that was previously stored in the user data database 900. The agent service 140 may ask questions to the user via the interaction service 150 and receives answers to these questions from the user through the interaction service 150, or the user may ask questions to the intelligent personal agents 145 in the agent service 140 via the interaction service 150.

As noted, the user interaction application 160 communicates with each of the various types of inputs and outputs 400, 500, 600, 700, 800 between a person, intelligent agent, or machine 200 and the platform 100, as described above in connection with FIG. 2. The user interaction application 160 communicates with the interaction service 150 to pass the inputs from the various external persons, intelligent agents, or machines 200 to the interaction service 150 for purposes of passing those inputs to the various components within the platform 100. In addition, the user interaction application 160 communicates with the interaction service 150 to pass the outputs from the various components within the platform 100 to the various external persons, intelligent agents, or machines 200.

The domain template database 180 comprises a set of domain specific concepts written in a programming language that can be used by the intelligent personal agent 145 to deliberate, perform actions, and communicate with other agents, people, or systems. A domain template can include knowledge about the various domain concepts, such as activities, actions, plans, artifact types and artifacts, location types and locations, and roles. In one embodiment, the domain template is written in the Brahms Agent Language. Accordingly, the domain template database 180 sends data to the agent service 140 and sends data to and receives data from the learning service 130 for the purpose of storing new or changed domain templates as described above in connection with the learning service 130 in FIG. 3.

In particular, the domain template database 180 stores a set of intelligent personal agent models for different domains or applications for which a user is using the platform. The domain template database 180 consists of a set of general model files written in the agent language that are used in every domain, and for each specific domain (such as remote patient monitoring or first responders support) the domain template database 180 has a set of domain-specific files written in the agent language. General templates have general rules and knowledge that are used by all intelligent personal agents. Domain-specific templates have domain specific rules and knowledge for intelligent personal agents. All intelligent personal agents 145 inherit from the general templates, but intelligent personal agents 145 directed to domain-specific domains only inherit the domain-specific templates from the domain that the domain-specific intelligent personal agent supports. It should be appreciated, however, that models that are created for a particular user may be added as templates to the domain template database 180 for use by other users as well.

Each template can consist of a number of files in different categories, such as for groups (i.e., roles or generalized agent templates), classes (i.e., general object templates), specific agents and objects, domain concepts, geography classes (types of geographical areas) and specific area objects. Domain templates are created based on modeling the knowledge for a particular domain in an agent domain language, which are created, for example, by a person (the domain modeler) that programs the domain template using the specific agent language.

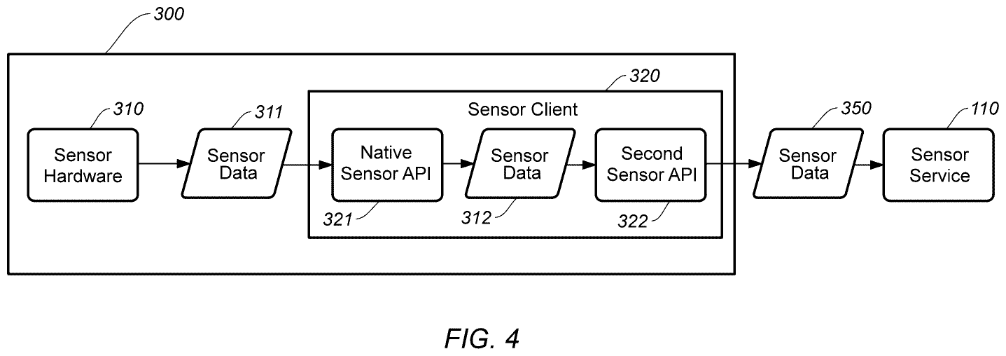

FIG. 4 is a block diagram illustrating the hardware and software components of an exemplary sensor for use in the intelligent personal agent platform and the corresponding data flow according to one embodiment of the present invention. The sensor 300 comprises several components, including sensor hardware 310 and sensor client 320. The sensor hardware 310 is physical hardware or a hardware platform that includes multiple physical sensors (e.g., mobile phone having, for example, accelerometer, gyroscope, proximity sensor, heart rate sensor, GPS, and a Bluetooth Low Energy sensor). The sensor hardware 310 collects sensor data 311 that is passed to the sensor client 320. The sensor client 320 is software to integrate sensors and serves as an interface between the sensor hardware 310 and the sensor service 110 within the platform 100 to allow data 311 from the sensor hardware 310 to be made compatible with and capable of being received by the sensor service 110. Accordingly, in one embodiment, the sensor client 320 can run on the external sensor 300, such as on a mobile phone, computer, cloud server, local server, or other hardware device. The sensor client 320 includes the native sensor Application Programming Interface (API) 321, which is an application interface that is developed by the sensor provider or which can be separately developed and which provides a way to capture sensor data 311 from the sensor hardware 310. The captured sensor data 311 from the sensor hardware 310 is passed from the native API 321 to a second API 322 within the sensor client 320. This second API 322 is a program that uses the sensor's native API 321 to query for data from the sensor hardware 310. The sensor data 350 retrieved from the native API 321 by the second API 322 is passed from the sensor 300 to the sensor service 110 within the platform 100.

FIG. 5 is a block diagram illustrating the agent service used in the intelligent personal agent platform 100 and data flow between the components of the agent service according to one embodiment of the present invention. The agent service 140 within the platform 100 comprises several components, including an agent manager 141, one or more intelligent personal agents 145, or one or more intelligent personal agents 145 for each user of the system, and one or more assistant agents 142 corresponding to one or more of the intelligent personal agents 145.

The agent manager 141 is an agent that creates and deletes intelligent personal agents 145 in the platform 100. Agents are created based on the data stored in the user data database 900. Each user specifies what type of role he or she plays within a given domain or application. For example, a user in a remote patient monitoring domain can play the role of the patient, the care provider, or the caregiver. Based on the user's role stored in the user data database 900, the agent manager 141 instantiates a type of agent in the agent service 140. For example, if the user plays the role of the patient, the agent manager 141 instantiates a patient agent. As noted above, in this case, the intelligent personal agent, which is the patient agent, would be obtained from the set of domain templates residing in the domain template database 180 for a patient monitoring domain and specifically the model for a patient.

The intelligent personal agent 145 has knowledge about the entity or user it serves and decides independently how to serve the user, depending on the domain or specific application for which the intelligent personal agent 145 is being used, its inputs, and its outputs. The intelligent personal agent 145 has knowledge, based on specific roles the agent performs, about the user that it uses to deliberate about the actions it needs to take. In other word, when the agent manager 141 creates the agent, it uses the particular domain template to get the knowledge for the agent type being generated. For example, the patient agent inherits this knowledge from the modeling of the patient role in the remote patient monitoring domain template. The agent manager instantiates the agents, objects, areas, and concepts as instances in the agent service. It should be appreciated that in some embodiments, an intelligent personal agent 145 may serve more than one user depending upon whether the intelligent personal agent 145 is using a general domain template or whether its domain-specific template can be used for more than one user.

The intelligent personal agent 145 can have one or more assistant agents 142 that it can ask assistance from to perform a specific task. The assistant agent 142 is an intelligent personal agent that can perform a specific task independently. As noted, the intelligent personal agent 145 can use multiple assistant agents in order to perform an overall high-level task. For example, one type of assistant agent may be an activity assistant agent. These are agents that provide agent task assistance for a particular activity, such as monitoring if the user is doing what is on the user's plan while doing a particular activity, such as taking his/her medication while having lunch. Another assistant agent may be a monitoring assistant agent. These are agents that monitor particular user data (e.g. particular incoming sensor data) and notify other agents (e.g. the intelligent personal agent 145) if something important occurs in or during the monitoring. For example, a heart rate monitoring assistant agent monitors the heart rate sensor data for a user and gives an alert when the heart rate is problematic based on rules described in that assistant agent. Another assistant agent may be a proxy agent. These are agents that provide a model and simulation of other agents. For example, a user proxy agent is an agent that simulates the user's behavior and predicts the user's activities at all times. Other agents can ask this agent for the user's current activity at any time. In another example, an assistant agent can be a dispatch agent that is responsible for receiving service requests from one type of agent (e.g., customer agents) and transmitting or assigning those requests to another type of agent (e.g., service provider agents). Assistant agents are either created by the agent manager 141, in a manner similar to the creation of an intelligent personal agent, if they are specified as particular agents in a given domain template or they can be created by agents already running in the agent service 140.

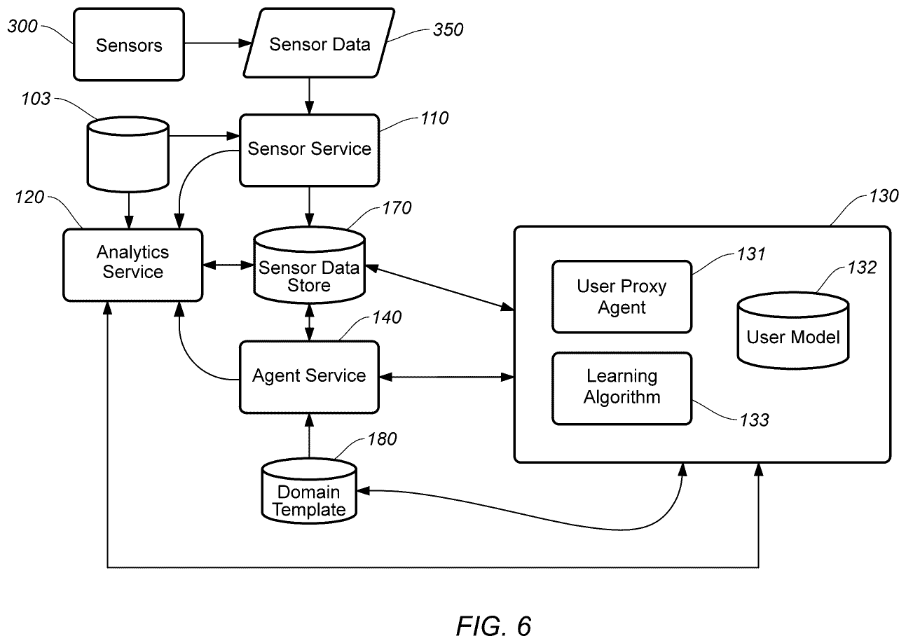

FIG. 6 is a block diagram illustrating the learning service used in the intelligent personal agent platform and data flow between the components of the learning service according to one embodiment of the present invention. Specifically, the learning service 130 comprises a user proxy agent 131, a user model 132, and a learning algorithm 133.

In one embodiment, the personal intelligent personal agent 145 can learn a user's behavior (e.g. learn John's most frequently visited locations in the past month) by calling a particular learning algorithm 133 in the learning service 130. The learning algorithm 133 may request data from the analytics service 120 or retrieve data from the sensor data store 170 (e.g., John's aggregated GPS coordinates for the past month). The learning algorithm 133 may also use a proxy agent 131 to simulate the user's behavior in the past or to predict future behavior. The user proxy agent 131 is a type of assistant agent 142 as described above in connection with FIG. 5 and is a software agent that simulates and predicts the user's behavior based on rules specified in a domain template 180 or a user model 132 (e.g., the proxy agent 131 applies rules such as inferring that John is at work when he is at a particular location during working hours or that he is at home when he is at a particular location during sleeping hours). The learning algorithm 133 then updates the user model 132 with the learned user behavior (e.g., it updates John's user model with the locations that John has frequented most in the last month). User models include databases that store user features that are learned for a particular user (e.g., the user's activities, health, location, or other measurements that are being observed or inferred for the user). User models can also include models that can be used to predict future behavior (predictive models). In one embodiment a predictive model can be written as set of rules (e.g., in the Brahms language). In another embodiment, a predictive model can be written as a probabilistic model, such as a Bayesian inference model or a Hidden Markov Model.

In some cases, the learning algorithm 133 can update the domain template 180 with the learned user behavior for a population of users (e.g., update the rules for a particular user role, such as a CHF patient, or a physician, or for all CHF patients at a particular hospital in San Francisco). By continually or periodically updating the user model 132, and the domain template model 180, more accurate models are created for the user, the user's role in the domain, and for a population of users over time. As noted, the domain template used by the intelligent personal agent 145 specifies the knowledge for the intelligent personal agent 145 to use to reason and act. Accordingly, as the domain template is modified by the updated user activity model 132 over time, the intelligent personal agent 145 becomes more knowledgeable as well and effectively learns.

In one embodiment, a personal intelligent personal agent 145 can make a prediction about the user's behavior, by calling a learning algorithm 133 to learn a prediction model 132 (e.g., a model to predict the next location a user will be in given his current location). The learning algorithm takes a user model 132 as input and learns a predictive model (e.g., a Hidden Markov Model of the probabilities of going from location A to location B). The personal intelligent personal agent 145 gets real time data from the analytics service 120 or sensor data store 170 (e.g., where the user is now, what activity the user is doing) as input and uses a user model 132 (e.g., a learned predictive model) and returns a prediction (e.g., where the user will go next, given the currently location and activity).

As noted above, the model used by the intelligent personal agent 145 may be selected from the set of templates stored in the domain template database 180. Alternatively, the model may be created by the learning service 130. In this embodiment, the learning service creates or generates a learned user model about some aspect related to a given user that can be used by an intelligent person agent 145 for that user. It should be appreciated, however, that once such a model is created, it may be stored in the domain template data base 180 for use as a template for other users or intelligent personal agents 145. Although a wide variety of such models may be created, following is a description of how such a model may be created. This specific example is directed to the creation of a model of a given user's important locations he or she frequents, as well as a learned user model of the user's schedule of when the user goes to these locations and how long the user is at each of these locations. For example, for a particular user the learning service learns the user's important location model by performing the following algorithm at a particular scheduled moment in time to improve the learned user model of the user's Important Locations model in the domain template database 180. The Location Learning algorithm 133 requests a timeline of the user's GPS positions for a period of time (e.g. 1 month) from the analytics service 120. To generate this timeline, the analytics service 120 clusters similar GPS coordinates. This is known as the aggregated location timeline. The location learning algorithm 133 takes the aggregated location timeline and applies a location-clustering algorithm in order to know how often a user visits a particular location. These are the clustered locations. Given the clustered locations, a \user proxy agent 131 now applies predefined behavioral rules about the type of locations a user would most likely be in at a particular moment of time (this is the location behavior template 132), which results in an improved model of important locations for the user in the domain template database 180. Next, given the new learned important locations, a prediction algorithm 133 applies a standard machine learning algorithm to learn a user location schedule model of when the user was at the important locations (e.g. using a learning algorithm to learn the Markov Model of going from location A to location B, to location C, etc. The learning service stores this in the user model database 132.

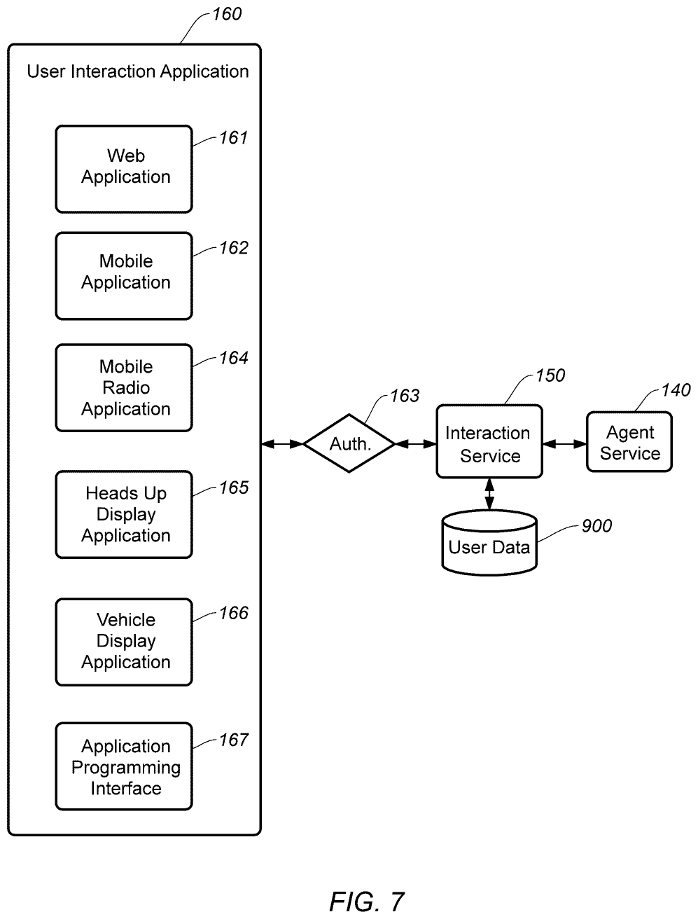

FIG. 7 is a block diagram illustrating the interaction service used in the intelligent personal agent platform and data flow between the components of the interaction service according to one embodiment of the present invention. The interaction service 150 is the service or software module that manages the interaction between the user and the platform 100.

The interaction service 150 allows for creation, deletion, and modification or updating of users. The interaction service 150 creates and deletes the intelligent personal agents 145 for the user, based on the information in the user data store database 900 (see also FIG. 3). It also manages the interaction between the user and his or her intelligent personal agents 145.

The interaction service 150 also enables the user and his or her intelligent personal agents to interact using any number of user interaction applications 160 (see FIG. 3) on a variety of devices. The interaction service 150 uses data supplied by the agent service 140 to track what device the user may be using (depending on context, location, etc.) to route information to the appropriate user interaction application 160 (e.g., to the mobile application running on the user's cell phone when a user is outside or to the car display application when a user is in the car). It also manages the display of data and information to the user interaction application 160.

For example, as shown in FIG. 7, the interaction service 150 communicates with various sample user interaction applications 161, 162, 164, 165, 166, 167 that allow a user to interact with the platform 100. These various user interaction applications can include a web application 161, which allows the user to interact with the platform 100 via a web browser; a mobile application 162, which allows the user to interact with the platform 100 on a mobile device such as a cell phone or tablet; a mobile radio application 164, which allows the user to interact with the platform 100 on a mobile radio or simply a radio; a heads-up display application 165, which allows the user to interact with the platform 100 via a heads-up display; a vehicle display application 166, which allows the user to interact with the platform 100 via a vehicle display; and an application programming interface 167, which allows the user to interact with the platform 100 via an API. Between the interaction service 150 and each of these user interaction applications 161, 162, 164, 165, 166, 167 there is an authentication process 163. The authentication process 163 is used to establish a connection to the interaction service 150, which is performed to identify, authenticate, or authorize the user and/or his device being used to interface with the platform 100. In this process, data is passed from the user or his device to the interaction service 150 where it is compared to data that is stored in the use data store database 900. This data may include something the user knows (e.g., password, PIN), something the user has in his possession (e.g., proof that he controls an email address, device UUID, software token) or something the user generates (biometrics, patterns of sensor data or activity that are generated by the user).

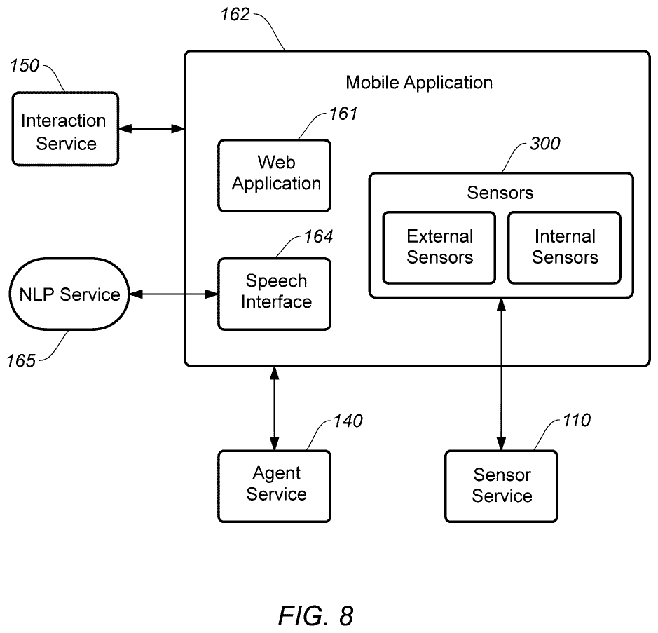

FIG. 8 is a block diagram illustrating a user interaction platform for interfacing with the intelligent personal agent platform according to one embodiment of the present invention. A user interaction application 160 (shown in FIG. 8 as a mobile application) runs on a user's device (e.g., phone, tablet, computer, watch, TV, car) that enables the user to interact with the device to enter input and receive output, for example via a graphical user interface, touch display, keyboard, mouse, gesture, or voice interaction.

In the embodiment shown in FIG. 8, a mobile application on a cell phone 162 allows users to interact with the platform 100 by voice interface and touch display on the cell phone. The mobile application 162 embeds a user interaction application (in this case shown as a web application 161) and also collects data from sensors 300 on the phone (or from other devices that are connected to the phone), which may include both external and internal sensors, which interface with the platform via the sensor service 110 (see FIG. 3). It should be appreciated that an external sensor is a sensor that is a sensor hardware platform external to the device on which the mobile application is executed. This sensor hardware platform connects and communicates to the device on which the mobile application runs via wired or wireless communication, e.g. Bluetooth. An internal sensor is a sensor hardware platform that is embedded in the device on which the mobile application runs. Internal sensor data is available to the mobile application from within the device's sensor application programming interface. Internal sensors are sensors that are embedded in the phone, such as accelerometers, gyroscope, or GPS. As noted, the system is capable to receive, collect, and monitor data from both external and internal sensors.

The interaction service 150 or the intelligent personal agents 145 running in the agent service 140 can send and receive data and notifications to and from the user via a screen or speech interface 164. Speech data that is received by the speech interface is sent to a natural language processing (NLP) service 165, which may be another application running on the device, an application running on the platform 100, or another third party service. The NLP service 165 translates speech data into processed or interpreted speech data.

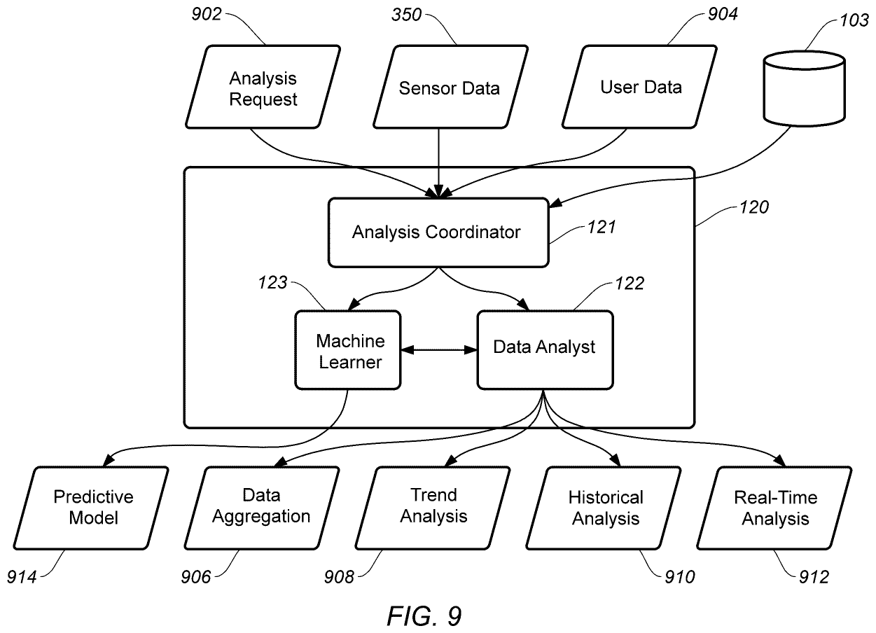

FIG. 9 is a block diagram illustrating the analytics service used in the intelligent personal agent platform and data flow between the components of the analytics service according to one embodiment of the present invention. As described above, the analytics service 120 comprises software that receives sensor data from the sensor data store database 170 that needs to be analyzed. More specifically, the analytics service 120 receives sensor data 350 from the sensor service 110, analysis requests 902 from the agent service 140, user data 904 from the interaction service 150, and any other data from the external database(s) 103 via the analysis coordinator 121. The analysis coordinator 121 determines what type of analytics is required for the received data and, based on this, sends the data and request to the data analyst 122 or the machine learner 123. The data analyst 122 can perform different analyses, including, for example, data aggregation 906, trend 908, historical 910, and real-time analyses 912. The machine learner 123 applies machine-learning algorithms known in the art to create predictive models 914 that can be used by the learning service 130.

The following is an example description of how the overall system operates for a given domain, in this case remote patient monitoring. The user, Hank, is a patient that is being monitored after he left the hospital with chronic heart failure (CHF). The hospital gave him a kit of sensors to take home, and he has some of his own devices at home or in his car, which he can link to the system. These may be passive sensors that stream continuous data (e.g., wearable heart rate monitor), sensors that trigger on some action (e.g., a door switch sensor that indicates that the refrigerator is opened), or sensors that require the user to take a measurement to acquire data (e.g., to put on a blood pressure cuff, step on a scale). Sensors may be in the home, car, or environment and measure physiology, use of an appliance, presence in a room, proximity to certain devices or people, environmental conditions, etc. The patient has an intelligent personal agent (in the agent service) that is continuously monitoring the patient. The agent uses sensor data from the sensor service, analytics data from the analytics service along with the template model and care plan (a plan with goals and tasks), and schedule to infer what the patient is doing now and what his health status is, predicting his activities and health status in the future, and analyzing how he is adhering to goals specified in his care plan. In addition, the patient's agent is connected to the intelligent personal agents of his caregivers (his family, friends, and health providers). Through the system, each member of his care team can monitor his activities and cooperate and coordinate to form a care team network. The various interactions and processes that the system effects in this remote patient monitoring domain example are described in more detail below.

According to his care plan, Hank is supposed to weigh himself at the same time each day (after waking and going to the bathroom, but before breakfast). When Hank is in the bathroom, his intelligent personal agent can send Hank a reminder to weigh himself, before he leaves the bathroom, which the agent knows is where the scale is located. If Hank needs to take a dose of medication with breakfast, the agent will remind him to take his medication when he is in the kitchen, as the intelligent personal agent has enough information to conclude that it is likely that he is making breakfast. The intelligent personal agent infers activity from the sensor data it receives (e.g., for example, it can infer that Hank is eating breakfast, based on the time of day, Hank's presence in kitchen, Hank's wrist worn sensors indicate he is eating and drinking, the refrigerator is opened, coffee pot is turned on). The appropriate display or interface is used depending on the context and devices that are available (e.g., a message sent to his watch or phone if he is outside, to his kitchen tablet, to the TV if he is in the living room, or car speakers if he is in the car, etc.).

Preferences can be set to create escalated notifications or actions for different events. For example, the reminders escalate until the intelligent personal agent infers that he has taken his medication (using sensors in pills or in pillboxes). First, he may get a visual reminder (a pillbox or light near the pillbox glows), but if he continues not to take the medication, then he gets a voice reminder over the house intercom system. If he still fails to take the mediation, a further escalation preference could be set to call Hank by phone. A further series of escalations can involve notifying others (e.g., his wife, then his daughter if wife is not available), health providers, or emergency services.

In addition, notifications can be sent to others for purposes of monitoring Hank's activities. For example, a friend of Hank's can ask to be notified in the future when Hank leaves his house and does not return within one hour. Alternatively, Hank can request that he be notified when this friend leaves their place of work and request that this friend call Hank. Frank's physician can request that she be notified when Frank's health status is observed or predicted to be deteriorating, or when Frank's deviates from goals or tasks specified in the care plan.

Further, anyone in the care team network could ask questions about the metrics that are being tracked and activities that are being inferred. These questions can be asked by text or voice and answered by text or voice or some other mechanism, depending on the devices being used to interact with the system (e.g. by formulating a text query or asking in natural language voice through the website, cell phone, microphone in home or car, or via a microphone embedded in a device, and the agent will respond by voice or text depending on the interface available to the user.). Hank's daughter might ask her own personal agent “How is Dad doing?” “Did he take his medication yet?” “Where is Dad?” “Did anyone visit Dad today?” How much time did that person spend with him?” etc. And the physician may ask his own personal agent “When did I change Hank's prescription?” “Did Hank gain weight the last time I increased the dosage of his medication”, or “How is Hank adhering to his medication schedule?”, etc. Frank's daughter's agent or the physician's agent may ask these question to Frank's agent, who will respond to the agent to the best of his knowledge based on its observations of Frank. The respective intelligent personal agent can then respond to its associated user based on the information it received from Frank's agent.

The intelligent personal agents can also use data analytics from the analytics service to create alerts or answer questions. Users can ask “is my weight appropriate?” and the intelligent personal agent will respond with the trend and likelihood of meeting a predefined goal, or the agent can help the user to come up with a plan to meet a defined goal.

The system can also send alerts when a simple threshold is reached (e.g., for a CHF patient, alert the physician when more than 5 pounds are gained in a week to detect fluid retention, which is an indication heart failure). In addition, the system may use the analytics service and a variety of analytic techniques (e.g., by analyzing historical data of weight data combined with physiological, social and environmental data for this individual or over a group of similar individuals) and learning and predictive techniques (as described above) to determine when an alert should be sent, rather than using a simple threshold. This may allow an earlier detection of an increased probability of risks, i.e. a prediction or indications of worsening problem, before the threshold is reached.

The intelligent personal agent can also ask questions of any of the users, e.g., “Hank, it looks like you are having shortness of breath? Are you feeling discomfort or pain?” Or, “Hank, have you taken your medication?” The answer is used to augment or improve the machine learning in the analytics service, which is referred to as “supervised machine learning” because the system can use the responses from humans to “label” or annotate the dataset that is used by the machine learning algorithms, and to improve the rules in the domain template or user models. For example, if an alert is sent for a patient that gains 2 lbs in a day, the agent can ask the physician if the alert was associated with a negative outcome for the patient, or if it was a “false positive” alert. Over time, the agents can learn, for example, that a 2 lb pound daily weight fluctuation is normal for a particular patient and can change the alert rule so that alerts are only sent if a patient gains over 3 lbs in a day.

The intelligent personal agent can also take action based upon a schedule, specified rules with conditions inferring what action to take, or requests from people or other agents. For example, the intelligent personal agent can order a car service, order medication from the pharmacy and have it delivered, schedule a meeting between multiple parties depending on joint availability, or coordinate and communicate information about to Hank to other people.

Further examples of specific situations regarding Hank's care illustrate the system's operation. For example, Hank and Jessica, his daughter, are walking outside, after having lunch. Hank's heart rate, respiration rate, GSR, etc., are being monitored through wearable sensors. The data from the sensors is sent to the system in the Cloud, where his intelligent personal agent is monitoring the sensor data and will provide alerts and support if something goes wrong. When Hank has a heart incident, the intelligent personal agent running in the Cloud notices this and will communicate to Hank and to Jessica to let them know that Hank is having a problem. The intelligent personal agent automatically schedules a car or, for example, an ambulance, to come to Hank and Jessica for further investigation. How the system works in this scenario is described below, with reference numbers corresponding to the Figures.

Hank 200 and Jessica 200 are walking outside after having lunch, when Hank experiences an irregular heart rate. His heart rate monitor 310 detects the heart rate signal and communicates the heart rate sensor data 350 to Hank's smart phone using Bluetooth. The mobile application 162 includes a heart rate Native Sensor API 321 that receives the heart rate sensor data 350. The heart rate Native Sensor API 321 sends this data to the sensor API 322 that translates this data into an API message containing the sensor data 350. Finally, the API message with sensor data 350 is send to the sensor service 110, where it is stored in the sensor data store database 170.

The sensor service 110 sends the received sensor data to the analytics service 120. The analytics service 120 determines that the heart rate data violates a threshold for Hank. The analytics service 120 sends a heart rate violation message with the heart rate data to Hank's intelligent personal agent 145. Hank's intelligent personal agent 145 infers, based on Hank's location information, activity, heart rate, and other physiological sensors, that this may be a sign of deterioration and that a car should be dispatched. The intelligent personal agent 145 schedules the car to go to Hank's location. It then infers that, because she is nearby Hank, Jessica should be called on her mobile phone. Hank's intelligent personal agent 145 running in the agent service 140 then schedules a car to pick up Hank.

Jessica receives a notification and phone call from her intelligent personal agent 145 that it has detected a car is nearby that can transport Hank to a clinic, emergency room, or provide additional point of care testing devices. Hank's intelligent personal agent 145 asks Hank via his mobile application 162 if he is ok. Hank answers the intelligent personal agent 145 via the speech interface 164. The speech interface 164 uses a NLP service 165 to analyze Hank's speech. The speech interface 164 receives the analyzed speech back from the NLP service 165 and creates an answer message to be sent to Hank's intelligent personal agent 145.

Jessica asks her intelligent personal agent 145 via her mobile application 162 how Hank is doing. The speech interface 164 uses a NLP service 165 to analyze Jessica's speech. The speech interface 164 receives the analyzed speech back from the NLP service 165 and creates a request message to be sent to Jessica's intelligent personal agent 145 running in the agent service 140. Jessica's intelligent personal agent 145 sends a request to Hank's intelligent personal agent 145 to ask for Hank's latest heart rate and activity. Hank's intelligent personal agent 145 sends the answer back to Jessica's intelligent personal agent 145, which in turn sends the reply message back to Jessica's mobile application 162. The speech interface 164 generates speech from the reply message it receives.

Once Hank is in the car, the system can continue to operate. In this situation, the car is integrated with medical sensors. The sensors are integrated with the Cloud. When the car arrives at Hank's location, Hank and Jessica both get into the car. The intelligent personal agent 145 calls in the physician and asks Hank to apply different medical sensor in the car. The sensor data is immediately sent for interpretation to the physician on call. How the system works is described below.

When Hank and Jessica get into the car, the proximity sensor in the car 310 is noticed via Bluetooth by the mobile application 300. The mobile application 300 includes a proximity sensor Native Sensor API 321 that receives the proximity sensor data 350. The proximity Native Sensor API 321 sends this data to the sensor API 322 that translates this data into an API message containing the sensor data 350. Finally, the API message with sensor data 350 is send to the sensor service 110, where it is stored in the sensor data store database 170.

The car has a pulse oximetry sensor to get blood oxygen saturation levels 310. This sensor data 350 is send to the application 300 in the car. The application includes a pulse oximetry Native Sensor API 321 that receives the pulse oximetry sensor data 350. The pulse oximetry Native Sensor API 321 sends this data to the sensor API 322 that translates this data into an API message containing the sensor data 350. Finally, the API message with sensor data 350 is send to the sensor service 110, where it is stored in the sensor data store database 170.

Various embodiments of the invention have been described above. However, it should be appreciated that alternative embodiments are possible and that the invention is not limited to the specific embodiments described above. In particular, while the invention has been described in the context of a given user, it should be appreciated that multiple users may use the system simultaneously. Similarly, it should be appreciated that the platform 100 has the capability of supporting multiple users and that the various templates in the template database 180 can be used to support multiple intelligent personal agents 145 to, in turn, support multiple users. Further, the platform 100 has the ability to thereafter learn about each of the multiple users and modify the models used by the intelligent personal agents 145 to provide a personal or tailored experience for each user.

Moreover, it should be appreciated that the present invention has virtually unlimited uses in that the system and methods can be used in many different domains or applications, such as supporting teams of people in the many different domains, such as healthcare, insurance, social networking, military, emergency response, patient monitoring, wellness, automotive, planning, scheduling, navigation, diagnosis, giving advice, support, etc. Accordingly, while the invention has been described above in connection with specific applications or domains or settings, such should not be viewed as limiting. For example, in the healthcare domain, the system allows sensor data to be collected from a patient's wearable sensors and sensors in the environment (e.g., in the car and home) in order to continuously monitor, learn and predict the health and behavior of a patient, wherever the patient may be, and allows actions to be taken (e.g., ordering medication, adjusting the room temperature, dynamically calculating the risk of hospital readmission, providing guidance, coaching, reminders, and alerts) to help the patient to achieve goals specified in their care plan, while allowing a team (e.g., the patient, caregivers, healthcare providers) to monitor the patient and to collaborate, coordinate and communicate when caring for the patient, wherever they may be. In the military domain, the system allows sensor data to be collected from a soldier's wearable sensors and sensors in the environment (e.g., in vehicle, in the field) in order to continuously monitor, learn and predict the health and behavior of the soldier, wherever he may be, and allows actions to be taken (e.g., displaying a map to support situational awareness, dynamically calculating a route to the nearest soldier, providing guidance, coaching, reminders, and alerts) to help the soldier to achieve goals specified in a mission plan, while allowing a team (soldier, platoon leader, commander, etc.) to monitor the soldier and to collaborate, coordinate and communicate in supporting the goals of the soldier, team, and mission. Similarly, in the domain of emergency responders, the system can be used in essentially the same way as in the military domain, where the first responders would be substituted for the soldiers. As another example, in the insurance domain, the system allows sensor data to be collected from an insured person's or insured property's wearable sensors and sensors in the environment (e.g., in the car and home) in order to continuously monitor, learn and predict the health and behavior of the insured person or property, wherever they may be, and allows actions to be taken (e.g., turning off an appliance, dynamically calculating the price of an insurance premium based on risk prediction, providing guidance, coaching, reminders, and alerts) to achieve goals specified in the insurance policy, while allowing a team (e.g., the insured person, his family members, the insurer, a service provider) to monitor the person or property, and to collaborate, coordinate and communicate in support of the insured person or property, wherever they may be.

What is claimed is:

1. A system for making predictions about a user, comprising:
a plurality of sensors for collecting real-time data about a user and the user's physical environment, wherein the plurality of sensors comprises at least one sensor carried by the user and at least one sensor physically disposed within the user's residence, and wherein the real-time data comprises at least one value of a physiological metric of the user; and
a plurality of artificially intelligent agents,
wherein a first agent in the plurality of artificially intelligent agents performs a first set of operations comprising:
obtaining the real-time data from the plurality of sensors,
communicating the obtained real-time data to a second agent in the plurality of artificially intelligent agents and to a third agent in the plurality of artificially intelligent agents,
wherein the second agent performs a second set of operations comprising:
analyzing the communicated real-time data to detect behavior patterns of the user, the behavior patterns comprising the user's physical activities and physical movements,
communicating the behavior patterns to a third agent in the plurality of artificially intelligent agents,
wherein the third agent performs a third set of operations comprising:
determining, based on the communicated behavior patterns and the communicated real-time data, the user's compliance with one or more pre-determined health goals, the one or more pre-determined health goals based on the physiological metrics,
wherein the determining the user's compliance comprises:
applying one or more rules to the real-time data to determine when one or more conditions have been met, and
deciding, when the one or more conditions have been met, that the user is not in compliance with the one or more pre-determined health goals,
wherein a fourth agent in the plurality of artificially intelligent agents performs a fourth set of operations comprising:
performing, when the user is not in compliance with the one or more goals, a response, and
wherein the performing the response further comprises:
making a prediction regarding the physiological metrics,
communicating to the user and at least one entity (i) the prediction regarding the physiological metrics, (ii) an alert that the user is not in compliance with the one or more pre-determined health goals, and (iii) a recommendation or request to the user to comply with the one or more pre-determined health goals.

2. The system of claim 1, wherein the performing the response further comprises requesting the at least one entity take an action based on the prediction, wherein the at least one entity comprises an individual other than the user or an intelligent agent.

3. The system of claim 2, wherein the communicating further comprises sending at least one of the prediction, the alert, and the recommendation to a user interface associated with one or more electronic devices, wherein the one or more electronic devices comprises a mobile computing device of the user and a mobile computing device of the individual.

4. The system of claim 3, wherein the one or more electronic devices further comprises a device containing the at least one sensor physically disposed within the user's residence.

5. The system of claim 4, wherein the real-time data further comprises physical locations associated with the user's physical activities and physical movements, and wherein the operations further comprise learning when and/or where the user performs the physical activities and physical movements.

6. The system of claim 5, wherein the physiological metrics comprise a heart rate of the user, a respiration rate of the user, and a blood oxygen saturation level of the user.

7. The method of claim 1, wherein said providing a response comprises contacting a person.

8. The system of claim 7, wherein the operations further comprise:
automatically scheduling the user's physical activities and/or physical movements to assist the user to comply with the one or more goals.

9. The system of claim 1, wherein the one or more rules and the one or more conditions are at least partially based on the real-time data.

10. The system of claim 9, wherein the first agent is operatively connected to a computing platform, the computing platform comprising an agent manager that creates and deletes one or more agents in the plurality of artificially intelligent personal agents.

11. A system for making predictions about a user, comprising:
a plurality of sensors for collecting real-time data about a user and the user's physical environment; and
a plurality of artificially intelligent agents comprising computer-executable instructions that, when executed, perform operations comprising:
obtaining a healthcare plan for the user, the healthcare plan comprising a set of actions to be taken by the user and a set of goals related to one or more physiological metrics of the user,
obtaining the real-time data from the plurality of sensors,
determining, by the real-time data, a plurality of physical locations visited by the user,
inferring, by the plurality of physical locations, a set of activities performed by the user,
when the set of activities fails to include at least one action in the set of actions, sending a reminder to the user to perform the at least one action,
determining, by the real-time data, the one or more physiological metrics of the user,
when the one or more physiological metrics of the user fails to meet at least one goal in the set of goals, sending an alert to the user, one or more individuals other than the user, and/or one or more agents in the plurality of artificially intelligent agents,
receiving one or more requests from the one or more individuals and/or the one or more agents to monitor the set of actions and/or the set of goals, and
sending, in response to the one or more requests, the reminder and/or the alert to the one or more individuals and/or the one or more agents,
wherein the plurality of sensors comprises:
a first sensor that continuously collects a first set of data in the real-time data and continuously sends the first set of data to the plurality of artificially intelligent agents,
a second sensor that collects a second set of data in the real-time data only when the user performs a triggering action, and
a third sensor that collects a third set of data in the real-time data only when the user uses a device that measures the one or more physiological metrics,
wherein the first sensor is worn and/or carried by the user, and wherein both the second sensor and the third sensor are physically disposed within the user's residence.

12. The system of claim 11, wherein the operations further comprise:
predicting, by the plurality of physical locations, a future time that the user will visit the plurality of locations; and
predicting, by the plurality of physical locations, the set of activities, and/or the one or more physiological metrics, a future value of the one or more physiological metrics.

13. The system of claim 12, wherein the plurality of physical locations comprises rooms within the user's residence, and wherein the real-time data comprises geographical proximity of the user to one or more objects within the user's residence.

14. The system of claim 13, wherein the reminder and/or the alert are communicated via a user interface associated with one or more electronic devices of the user, wherein the one or more electronic devices comprises a mobile computing device of the user, an electronic appliance of the user, and an electronic interface in a vehicle.

15. The system of claim 14, wherein the plurality of sensors further comprises additional sensors physically disposed outside of the user's residence, and wherein the additional sensors comprise a sensor physically disposed within the vehicle.

16. A system for making predictions about a user, comprising:
a plurality of sensors for real-time collection of data about a user, the data comprising physiological metrics of the user;
a plurality of artificially intelligent agents,
wherein at least one agent in the plurality of artificially intelligent agents performs operations comprising:
obtaining the data from the plurality of sensors,
analyzing the data to detect behavior patterns of the user, the behavior patterns comprising at least one physical environment in which the user is located,
determining, by the behavior patterns, the physiological metrics of the user,
when the physiological metrics of the user fails to meet at least one pre-determined health goal of the user, performing a response,
wherein the plurality of sensors comprises:
a first sensor in physical contact with the user,
a second sensor physically disposed within the at least one physical environment,
a third sensor that begins the real-time collection of data only when the user uses a device, and
wherein each of the plurality of artificially intelligent personal agents:
communicates with at least one other agent in the plurality of artificially intelligent personal agents, the communicating comprising sending and/or receiving at least one communicative act,
are decentralized such that each of the plurality of artificially intelligent personal agents is not connected to a cloud-based server.

17. The system of claim 16, wherein the at least one physical environment comprises an enclosed, interior area around the user.

18. The system of claim 17, wherein the enclosed, interior area is selected from the group consisting of: a building, a vehicle, and combinations thereof.

19. The system of claim 15, wherein the performing the response comprises:
sending an alert to the user that the user is not in compliance with the at least one pre-determined health goal; and
sending a recommendation to the user to assist the user to comply with the at least one pre-determined health goal.

20. The system of claim 15, wherein the physiological metrics comprise at least one of: a heart rate of the user, a respiration rate of the user, and a blood oxygen saturation level of the user, wherein the behavior patterns further comprise the user's physical activities and physical movements, and wherein the operations further comprise:
learning when the user performs the physical activities and the physical movements,
asking the user at least one question regarding the physiological metrics, and
receiving, from the user, at least one answer to the at least one question regarding the physiological metrics.
